(19) United States
(12) Patent Application Publication
(10) Pub. No.: US 2013/0336961 A1
(43) Pub. Date: Dec. 19, 2013

(54) B Cell Depletion for Central Nervous System Injuries and Methods and Uses Thereof

(71) Applicants: Phillip G. Popovich, Columbus, OH (US); Daniel P. Ankeny, New York, NY (US)

(72) Inventors: Phillip G. Popovich, Columbus, OH (US); Daniel P. Ankeny, New York, NY (US)

(73) Assignee: THE OHIO STATE UNIVERSITY, Columbus, OH (US)

(21) Appl. No.: 13/810,559

(22) PCT Filed: Jul. 15, 2011
(86) PCT No.: PCT/US11/44192

§ 371 Date: Aug. 29, 2013

Related U.S. Application Data
(60) Provisional application No. 61/365,057, filed on Jul. 16, 2010.

(51) Int. Cl. A61K 39/395

(52) U.S. Cl. CPC A61K 39/3955
USPC 424/133.1; 424/173.1; 424/144.1

(57) ABSTRACT

Therapeutic B lymphocyte (B cell) depleting antibodies and methods and uses thereof in the treatment of patients having central nervous system injuries are described.

### CROSS-REFERENCE TO RELATED APPLICATIONS

This is a national stage application filed under 37 CFR 1.371 of international application PCT/US______ filed ______ which claims the priority to U.S. Provisional Application Ser. No. 61/365,057 filed Jul. 16, 2010, the entire disclosures of which are expressly incorporated herein by reference.

### STATEMENT REGARDING SPONSORED RESEARCH

This invention was made with government support under Grant number NIH R01 NSO47175 and NIH R03 NS055871. The government has certain rights in this invention.

### FIELD OF INVENTION

In a broad aspect, the present invention relates to therapeutic B lymphocyte (B cell) depleting antibodies and methods and uses thereof in the treatment of patients having central nervous system injuries.

### BACKGROUND OF THE INVENTION

There is no admission that the background art disclosed in this section legally constitutes prior art.

Traumatic injury to the mammalian spinal cord activates B lymphocytes (B cells) culminating in the synthesis of autoantibodies. The functional significance of this immune response is unclear. The consequences of neuroinflammation caused by spinal cord injury (SCI) have been inferred mostly from studies manipulating the function or survival of neutrophils, monocytes/macrophages or T lymphocytes (T cells). Less is known about the role played by antibody-producing B cells.

Spinal cord injury (SCI) triggers immune responses that can simultaneously exacerbate tissue injury and promote central nervous system (CNS) repair. After SCI, B cells produce antibodies that bind CNS and non-CNS antigens. This component of the immune response exacerbates pathology caused by SCI.

Revealing the identity and source of these antibodies is highly desirable since it is clear that not all antibodies are pathogenic and even those with this capacity may trigger repair at lower concentrations.

Until the inventors' latest discovery (which forms the basis of at least a part of the present invention), a causal role for B cells as effectors of post-SCI pathology has not been proven. Described herein is this latest discovery by the inventors that provides methods and uses of antibodies in the treatment of patients having CNS injuries.

### SUMMARY OF THE INVENTION

In a first broad aspect, there is provided herein use of a therapeutic B lymphocyte (B cell) depleting antibody in a patient in need thereof to block B cell-mediated pathology in human or animal neurological disorders.

In certain embodiments, B cells are depleted via infusion of anti-CD20 antibodies. In one embodiment, the anti-CD20 antibodies are selected from Rituximab or Ocrelizumab.

In certain embodiments, B cells are depleted via infusion of a combination of anti-CD79alpha and anti-CD79beta antibodies.

In certain embodiments, the human or animal neurological disorders include traumatic brain or spinal cord injuries, spinal ischemia, stroke, Alzheimer's disease, Parkinson's disease, and schizophrenia.

In another broad aspect, there is provided herein use of a B lymphocyte (B cell) therapy for lessening the severity of tissue damage and for restoring locomotor function in a patient in need thereof.

In a further broad aspect, there is provided herein use of a B cell depleting antibody for the manufacture of a medicament for the treatment of a neurological disorder.

In certain embodiments, the neurological disorder is in a subject having a traumatic brain or spinal cord injury, spinal ischemia, stroke, Alzheimer's disease, Parkinson's disease, or schizophrenia.

In certain embodiments, the B cell depleting antibody is an anti-CD20 antibody.

In certain embodiments, the B cell depleting antibody is selected from rituximab and antibodies directed against B cell surface molecules, more preferably antibodies directed against B cell specific surface molecules, such as CD20.

In certain embodiments, the B cell depleting antibody is administered in a dose of 1 mg to 1 g, preferably 100 mg to 800 mg, more preferably 250 mg to 750 mg, most preferably 300 mg to 500 mg.

In certain embodiments, the B cell depleting antibody is administered in one dose every 2-20 days, preferably one dose every 7-14 days.

In certain embodiments, the B cell depleting antibody is administered in one dose every 1-3 days.

In certain embodiments, the B cell depleting antibody is administered in 1-20 doses in total, preferably in 1-10 doses, more preferably 1-8 doses, and most preferably 1-4 doses in total.

In certain embodiments, the administration is systemical, preferably via injection or infusion, more preferably an intravenous injection or infusion.

In certain embodiments, the B cell depleting antibody is administered to a subject in need thereof, and the administration results in a prevention of a deterioration of neurological function.

In certain embodiments, the B cell depleting antibody is administered prior to or after a different treatment modality.

In certain embodiments, the B cell depleting antibody is administered in combination with other medication.

In another broad aspect, there is provided herein a method treating a patient with a neurological disorder. The method includes providing a therapeutic B lymphocyte (B cell) depleting antibody to block B cell-mediated pathology in the patient. The method further includes depleting B cells via infusion of antibodies in the patient to lessen the severity of tissue damage and to restore locomotor function in the patient.

In certain embodiments, B cells are depleted via infusion of anti-CD20 antibodies. In one embodiment, the anti-CD20 antibodies are selected from Rituximab or Ocrelizumab.

In certain embodiments, B cells are depleted via infusion of a combination of anti-CD79alpha and anti-CD79beta antibodies.

In a further broad aspect, there is provided herein a method of treating a neurological disorder. The method includes administering to a subject in need thereof effective amounts of an anti-CD20 antibody such that administration of the anti-CD20 antibody provides a synergistic improvement in the incidence or symptoms of a neurological disorder.

In certain embodiments, the anti-CD20 antibody is a non T cell depleting antibody.

In certain embodiments, the anti-CD20 antibody is a humanized antibody.

In a still further broad aspect, there is provided herein a method of treating a subject suffering from or predisposed to a neurological disorder. The method includes administering a therapeutically effective amount of at least one B cell depleting antibody to the subject.

In certain embodiments, the B cell depleting antibodies are monoclonal antibodies.

In certain embodiments, the monoclonal antibodies are selected from chimeric antibodies and humanized antibodies.

In certain embodiments, the neurological disorder is selected from traumatic brain or spinal cord injuries, spinal ischemic, stroke, Alzheimer's disease, Parkinson's disease, and schizophrenia.

In certain embodiments, the B cell depleting antibody reacts with or binds to a CD20 antigen.

In certain embodiments, the B cell depleting antibody is Rituximab and/or Ocrelizumab.

In another broad aspect, there is provided herein a method of treating a subject suffering from or predisposed to a neurological disorder. The method includes administering a therapeutically effective amount of at least one immunoregulatory antibody to the subject such that the immunoregulatory antibody binds to an antigen selected from CD79alpha, CD79beta and CD20 antigens.

In certain embodiments, the immunoregulatory antibody is a monoclonal antibody.

In certain embodiments, the monoclonal antibody is selected from chimeric antibodies and humanized antibodies.

In certain embodiments, the neurological disorder is selected from traumatic brain or spinal cord injuries, spinal ischemic, stroke, Alzheimer's disease, Parkinson's disease, and schizophrenia.

In certain embodiments, the method further includes the step of administering a B cell depleting antibody.

In certain embodiments, the B cell depleting antibody reacts with or binds to CD20 antigen.

In certain embodiments, the B cell depleting antibody reacts with or binds to CD79alpha antigen.

In certain embodiments, the B cell depleting antibody reacts with or binds to CD79beta antigen.

In certain embodiments, the B cell depleting antibody reacts with or binds to a combination of CD79alpha and CD79beta antigens.

Other methods, uses, features, and advantages of the present invention will be or will become apparent to one with skill in the art upon examination of the following drawings and detailed description. It is intended that all such additional methods, uses, features, and advantages be included within this description, be within the scope of the present invention, and be protected by the accompanying claims.

### BRIEF DESCRIPTION OF THE DRAWINGS

FIGS. 1A-1B are exemplary schematic graphs showing recovery from spinal cord injury in BCKO and WT mice in which locomotor function is analyzed using BMS score and subscore analyses, respectively.

FIGS. 2A-2C are exemplary schematic bar graphs showing total lesion volume and spared spinal cord gray matter (GM) and white matter (WM) in BCKO and WT mice.

FIGS. 2D-2E are exemplary images of three-dimensional reconstructions of spinal cords taken from WT mouse and a BCKO mouse.

FIGS. 2F-2G are exemplary images of immunofluorescent double-labeling of spared white matter (WM) from a WT mouse and a BCKO mouse.

FIG. 3A is an exemplary schematic graph showing ELISA analysis of cerebrospinal fluid from WT and BCKO mice.

FIGS. 3B-3C are exemplary schematic graphs showing quantitative analysis of intraspinal B cell accumulation at 28 days post-injury and the proportional area of IgG staining as a function of time post-SCI, respectively, at the site of injury in various strains of mice.

FIG. 3D is a series of images of representative sections from uninjured BL/6 or SCI BCKO and WT mice revealing the specificity of IgG labeling quantified in FIG. 3.

FIG. 3E is an exemplary flattened confocal z-stack image revealing accumulation of endogenous antibodies and Ig+B cells in the injured spinal cord.

FIG. 3F is an exemplary flattened z-stack image with x, y, z-projections showing B220-negative plasma cells with IgG cytoplasm (arrows) co-localized with, but distinct from IgG⁺B220⁺ B cells (arrowheads).

FIG. 4A is an exemplary sequence of still video images one day after injecting naïve (uninjured) mice with control (uninjured) showing one complete step cycle.

FIG. 4B is an exemplary sequence of still video images one day after injecting naïve (uninjured) mice with SCI antibodies showing one complete step cycle.

FIG. 4C is an exemplary schematic graph showing a summary of hind limb function in injected naïve mice with control or SCI antibodies ipsilateral to the injection site.

FIG. 4D is a set of exemplary low (upper box) and high power (lower box) images from a mouse injected with control with asterisk indicating injection target.

FIG. 4E is a set of exemplary low (upper box) and high power (lower box) images from a mouse injected with SCI antibodies with asterisk indicating injection target.

FIG. 4F is an exemplary image of phagocytic and microglia/macrophages co-localized with axon/neuron pathology at the site of injection in mice receiving SCI antibodies.

FIGS. 4G-4I are exemplary high power images of boxed region shown in FIG. 4F.

FIG. 5A is an exemplary schematic graph showing a summary of function in hind limb of mice ipsilateral to the site where purified control or SCI antibodies were injected.

FIGS. 5B-5C are exemplary schematic bar graphs showing the total SC volume and lesion volume, respectively, in various strains of mice.

FIG. 5D are exemplary images of three-dimensional reconstructions showing the pathology caused by injections of SCI antibodies into WT, C3^{-/-}, or FcR^{-/-} mice.

FIGS. 6A-6F are representative images showing IgG and complement Clq co-localized in regions of pathology in spinal cord of WT mice (FIGS. 6A, 6C, and 6E-6F) and BCKO mice (FIGS. 6B and 6D).

FIG. 7A is an exemplary schematic bar graph showing quantitative analysis of spared white matter (SWM) at the epicenter at 63 dpi.

FIGS. 7B-7C are immunofluorescent double-labeled representative images from WT and BCKO mice, respectively, showing axons and myelin in the epicenter.

FIGS. 7D-7G are exemplary schematic graphs showing a rostral-caudal distribution of total tissue, total lesion, and spared white and gray matter in WT and BCKO mice.

FIGS. 8A-8E are exemplary schematic graphs showing isotype-specific ELISA revealing differential production of different antibody isotypes at 63 dpi in C57BL/6 WT mice.

FIGS. 8F-8G are exemplary schematic bar graphs showing ELISA data comparing total Ig levels in rat serum 42 days post-injury or sham injury.

FIGS. 9A-9B show characterization of antibodies purified from sera of SCI mice in which identical volumes were loaded into each lane prior to SDS-PAGE and western blotting. FIG. 9A is an exemplary gel showing coomassie-staining of total proteins in the gel prior to transfer.

FIG. 9B is an exemplary western blot showing anti-mouse IgM+IgG staining of the membrane post-transfer.

FIGS. 10A-10B are exemplary schematic graphs showing a rostral-caudal distribution of total tissue (cross-sectional areas) and fraction of section occupied by lesion, respectively, in WT, C3^{-/-} or FcγR^{-/-} mice receiving microinjections of purified control or SCI antibodies.

### DETAILED DESCRIPTION OF THE PREFERRED EMBODIMENT(S)

Throughout this disclosure, various publications, patents and published patent specifications, are referenced by an identifying citation. The disclosures of these publications, patents and published patent specifications, are hereby incorporated by reference into the present disclosure to more fully describe the state of the art to which this invention pertains.

In one aspect, there is provided herein use of a therapeutic B lymphocyte (B cell) depleting antibody in a patient in need thereof to block B cell-mediated pathology in human or animal neurological disorders.

In certain embodiments, B cells are depleted via infusion of anti-CD20 antibodies. In one embodiment, the anti-CD20 antibodies are selected from Rituximab or Ocrelizumab. Non-limiting examples of anti-CD20 antibodies include Rituximab, Ocrelizumab,

It should be understood that other appropriate anti-human antibodies, including those currently in development, may be used in conjunction with the present invention.

In certain embodiments, B cells are depleted via infusion of a combination of anti-CD79alpha and anti-CD79beta antibodies.

In certain embodiments, the human or animal neurological disorders include traumatic brain or spinal cord injuries, spinal ischemia, stroke, Alzheimer's disease, Parkinson's disease, and schizophrenia.

In another aspect, there is provided herein use of a B lymphocyte (B cell) therapy for lessening the severity of tissue damage and for restoring locomotor function in a patient in need thereof.

In a further broad aspect, there is provided herein use of a B cell depleting antibody for the manufacture of a medicament for the treatment of a neurological disorder.

In certain embodiments, the neurological disorder is in a subject having a traumatic brain or spinal cord injury, spinal ischemia, stroke, Alzheimer's disease, Parkinson's disease, or schizophrenia.

In certain embodiments, the B cell depleting antibody is an anti-CD20 antibody.

In certain embodiments, the B cell depleting antibody is selected from rituximab and antibodies directed against B cell surface molecules, more preferably antibodies directed against B-cell specific surface molecules, such as CD20.

In certain embodiments, the B cell depleting antibody is administered in a dose of 1 mg to 1 g, preferably 100 mg to 800 mg, more preferably 250 mg to 750 mg, most preferably 300 mg to 500 mg.

In certain embodiments, the B cell depleting antibody is administered in one dose every 2-20 days, preferably one dose every 7-14 days.

In certain embodiments, the B cell depleting antibody is administered in one dose every 1-3 days.

In certain embodiments, the B cell depleting antibody is administered in 1-20 doses in total, preferably in 1-10 doses, more preferably 1-8 doses, and most preferably 1-4 doses in total.

In certain embodiments, the administration is systemical, preferably via injection or infusion, more preferably an intravenous injection or infusion.

In certain embodiments, the B cell depleting antibody is administered to a subject in need thereof, and the administration results in a prevention of a deterioration of neurological function.

In certain embodiments, the B cell depleting antibody is administered prior to or after a different treatment modality.

In certain embodiments, the B cell depleting antibody is administered in combination with other medication.

In another broad aspect, there is provided herein a method treating a patient with a neurological disorder. The method includes providing a therapeutic B lymphocyte (B cell) depleting antibody to block B cell-mediated pathology in the patient. The method further includes depleting B cells via infusion of antibodies in the patient to lessen the severity of tissue damage and to restore locomotor function in the patient.

In certain embodiments, B cells are depleted via infusion of anti-CD20 antibodies. In one embodiment, the anti-CD20 antibodies are selected from Rituximab or Ocrelizumab.

In certain embodiments, B cells are depleted via infusion of a combination of anti-CD79alpha and anti-CD79beta antibodies.

In a further broad aspect, there is provided herein a method of treating a neurological disorder. The method includes administering to a subject in need thereof effective amounts of an anti-CD20 antibody such that administration of the anti-CD20 antibody provides a synergistic improvement in the incidence or symptoms of a neurological disorder.

In certain embodiments, the anti-CD20 antibody is a non T cell depleting antibody.

In certain embodiments, the anti-CD20 antibody is a humanized antibody.

In a still further broad aspect, there is provided herein a method of treating a subject suffering from or predisposed to a neurological disorder. The method includes administering a therapeutically effective amount of at least one B cell depleting antibody to the subject.

In certain embodiments, the B cell depleting antibodies are monoclonal antibodies.

In certain embodiments, the monoclonal antibodies are selected from chimeric antibodies and humanized antibodies.

In certain embodiments, the neurological disorder is selected from traumatic brain or spinal cord injuries, spinal ischemia, stroke, Alzheimer's disease, Parkinson's disease, and schizophrenia.

In certain embodiments, the B cell depleting antibody reacts with or binds to a CD20 antigen.

In certain embodiments, the B cell depleting antibody is Rituximab and/or Ocrelizumab.

In another broad aspect, there is provided herein a method of treating a subject suffering from or predisposed to a neurological disorder. The method includes administering a therapeutically effective amount of at least one immunoregulatory antibody to the subject such that the immunoregulatory antibody binds to an antigen selected from CD79alpha, CD79beta and CD20 antigens.

In certain embodiments, the immunoregulatory antibody is a monoclonal antibody.

In certain embodiments, the monoclonal antibody is selected from chimeric antibodies and humanized antibodies. It should be understood that any appropriate chimeric and human antibodies, including those currently in development, may be used in conjunction with the present invention.

In certain embodiments, the neurological disorder is selected from traumatic brain or spinal cord injuries, spinal ischemia, stroke, Alzheimer's disease, Parkinson's disease, and schizophrenia.

In certain embodiments, the method further includes the step of administering a B cell depleting antibody.

In certain embodiments, the B cell depleting antibody reacts with or binds to CD20 antigen.

In certain embodiments, the B cell depleting antibody reacts with or binds to CD79alpha antigen.

In certain embodiments, the B cell depleting antibody reacts with or binds to CD79beta antigen.

In certain embodiments, the B cell depleting antibody reacts with or binds to a combination of CD79alpha and CD79beta antigens.

Spinal Cord Injuries

Spinal cord injury (SCI) potently activates B cells, culminating with increased synthesis of autoantibodies. There is a seemingly paradoxical effect of SCI on B cell function, i.e., complete spinal cord transection at high thoracic levels (T3) induces marked apoptosis of splenic B cells within 72 hours post-injury. Experimental immunizations during this acute post-injury interval fail to elicit antibody synthesis. This phenomenon appears to be level-dependent as neither complete spinal transection nor incomplete spinal contusion injury at mid-thoracic level (T9) causes significant B cell apoptosis. On the contrary, T9 spinal contusion activates B cells within 24 hours post-injury causing them to secrete IgM then IgG antibodies. Then, after about 14 days, significant levels of autoantibodies are detected in T9 SCI mice. Despite the rapid onset of acute immune suppression in this model, B cell function, including synthesis of autoantibodies, may be restored at later times post-injury. Indeed, although injuries at cervical or lower spinal levels in humans cause immune suppression, high titers of CNS-reactive autoantibodies also exist in these individuals. Additional functions of B cells, including their potential role as antigen presenting cells or immune-regulatory cells, should also be considered as these functions may predominate during the acute phase of injury (less than 14 days post-injury), i.e., before significant production of autoantibodies is detectable.

Clq binds antigen-antibody (immune) complexes, which initiates enzymatic conversion of other complement proteins. The result is formation of a lytic membrane attack complex and recruitment/activation of myeloid lineage cells (e.g., microglia/macrophages) that bear complement receptors. Using a model of spinal contusion injury, there was observed marked neuroprotection in mice that could not make immune complexes (i.e., BCKO mice); these mice had minimal Clq deposition at/nearby sites of SCI. Conversely, in SCI WT mice, intraspinal antibody deposits co-localize with Clq labeling. Thus, delayed accumulation of intraspinal antibodies causes pathology in part by activating complement. Recently, a deficiency in Clq was shown to be neuroprotective and promote functional recovery after SCI or traumatic brain injury.

Injection of pathogenic SCI antibodies into the spinal cord of complement deficient mice is benign relative to that caused by identical injections into wild-type (WT) spinal cord. Injection of SCI antibodies into FcR-deficient mice produced similarly mild injuries that may show SCI antibodies also initiate inflammation by ligating Fc receptors on macrophages, microglia or other FcR-bearing immune cells.

Despite the delayed and chronic accumulation of intraspinal B cell clusters and autoantibodies, a decline in locomotor function does not occur at later times post-injury in WT mice. Instead, functional recovery plateaus. While not wishing to be bound by theory, the inventors herein believe that this may indicate that antibodies are antagonizing mechanisms of endogenous repair rather than causing direct toxicity to cells that area unaffected by the primary trauma. For example, the inventors have evidence that autoantibodies specific for proteins in axonal growth cones are increased after SCI. Antibody binding to growth cones could block axonal plasticity and/or long tract regeneration. Post-SCI elevations of anti-endothelial antibodies may also thwart the repair of the microvasculature thereby limiting the supply of oxygen and nutrients to the spinal parenchyma.

As antibody levels rise and parallel inflammatory cascades are initiated, antibodies cause pathology; even monomeric IgG becomes pathologic at high concentrations. In particular, BCKO mice show progressive functional recovery with reduced pathology at times when B cell activation and antibody synthesis appear to limit recovery levels in WT mice. Since the sterile inflammation and tissue destruction caused by intraspinal injection of SCI antibodies was mitigated in complement or FcR deficient mice, these mechanisms of immunity are activated downstream of B cell activation and antibody synthesis after SCI. IgG/Clq deposits co-localize near sites of tissue injury, including areas of visible neuron pathology, and functional recovery is improved in complement-deficient mice. A further explanation for the inventors' results is that the post-injury rise in circulating and CSF antibodies is a mechanism of protein homeostasis. In acute post-streptococcal glomerulonephritis, circulating IgG levels rise precipitously but without a proportional change in immune complex formation in the kidney. This is of interest since pathogenic antibodies cause kidney pathology in this disease. High titers of IgG may assist in “buffering” pathogenic proteins, including antibodies, while minimizing the loss or degradation of proteins (e.g., albumin) that are depleted during the course of the disease.

As shown in the Example set forth below, the present data reveal an unexpected role for B cells and antibodies as effectors of pathology after SCI. Various self-antigens are released or become altered by SCI causing B cell activation and secretion of antibodies that trigger pathogenic complement cascades and microglia/macrophages.

### Example

The present invention is further defined in the following Example, in which all parts and percentages are by weight and degrees are Celsius, unless otherwise stated. It should be understood that this Example, while indicating preferred embodiments of the invention, is given by way of illustration only. From the above discussion and this Example, one skilled in the art can ascertain the essential characteristics of this invention, and without departing from the spirit and scope thereof, can make various changes and modifications of the invention to adapt it to various usages and conditions. All publications, including patents and non-patent literature, referred to in this specification are expressly incorporated by reference herein.

### EXPERIMENTAL

### Materials and Methods

Mice

Specific pathogen-free C57BL/6J [wild-type (WT), n=28], IgH-6 [B cell knockout (BCKO), n=17)], and B6.12954-C3tmlCrr/J [complement component C3 knockout (C3^{-/-}), n=4] mice were obtained from The Jackson Laboratory (Bar Harbor, Me.). Fcerlg [FcRγ knockout mice (FcR^{-/-}), n=6] were obtained from Taconic Farms (Hudson, N.Y.). BL 10, B10.PL and Balb/cJ mice were obtained from Harlan Labs (Indianapolis, Ind.). All mice were females, age 7-8 weeks and weighed 17-22 g at the time of surgery. All mice were housed in HEPA-filtered Bio-clean units in a sterile room (barrier housing). All procedures were approved by and performed in accordance with The Ohio State University's Institutional Lab Animal Care and Use Committee.

Spinal Cord Injury

Mice received a mid-thoracic spinal contusion injury using the Ohio State University electromechanical spinal contusion device. Briefly, mice were anesthetized with ketamine and xylazine (80 mg/kg and 10 mg/kg respectively, i.p.), then given prophylactic antibiotics (Gentocin; 1 mg/kg, s.q.). Using aseptic technique, a partial vertebral laminectomy was performed at the mid-thoracic level (T₉₋₁₀). The exposed dorsal spinal surface (T₉ spinal level) was displaced a calibrated vertical distance (0.5 mm over about 25 ms), producing a moderately severe spinal cord injury. After surgery, muscles and skin were sutured and mice hydrated with physiological saline (2 ml, s.q.). Bladders were voided manually 2×/day and hydration was monitored daily and urinary pH monitored weekly. All animals were within normal parameters of displacement, force and impulse-momentum (Grubb's test to detect outliers; t-test comparing biomechanic parameters between groups or individual strains yielded p-values>0.72 for all measures).

Behavioral Analysis—BMS Test and Subscore

Locomotor recovery was compared using the BMS locomotor rating scale specifically designed for use in SCI mice. The BMS is a 10-point scale based on operational definitions of hind limb movement with additional emphasis placed on evaluating trunk stability. Briefly, individual mice were simultaneously observed by two investigators for a four-minute testing period, during which hind limb movements, trunk/tail stability and forelimb-hindlimb coordination were assessed then graded according to published methods. The subscore component of the BMS scores individual aspects of fine locomotor control for the hindlimbs (e.g., paw rotation at initial contact) and trunk/tail (e.g., ability to maintain tail in upright position during locomotion), e.g., aspects of locomotion that if they were to change alone they would not necessarily change the overall BMS score.

Cerebrospinal Fluid Collections

Cerebrospinal fluid (CSF) was collected from anesthetized mice at 63 dpi via the cisterna magna. Briefly, a sharpened 0.5 μL Hamilton syringe was used to puncture then aspirate about 10 μL of clear CSF. Mice were then immediately perfused as outlined below. CSF samples were transferred to individual tubes and frozen at −80° C. Total Ig levels were measured by ELISA.

### Experimental

### Antibody Microinjection Studies

Serum Collection

Whole blood was obtained from awake, unrestrained mice via retro-orbital puncture. Samples were collected from SCI mice before injury and at 42 days post spinal cord injury and from uninjured mice at 42 days after laminectomy (see below).

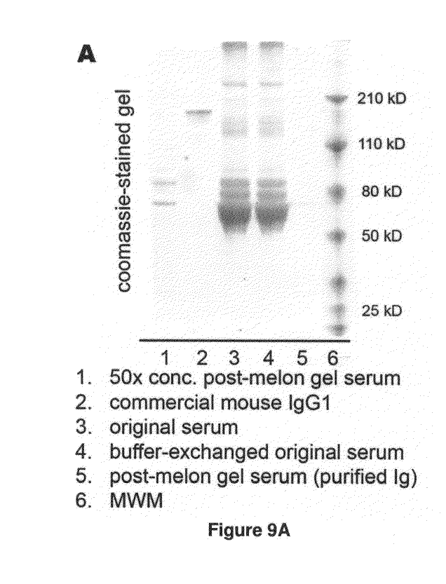

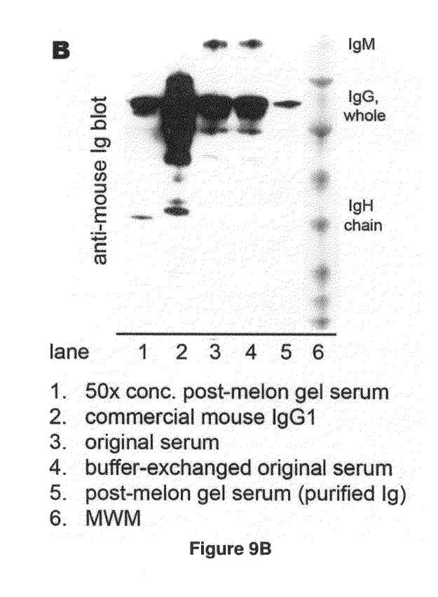

Purification of Antibodies from SCI and Uninjured Serum Total serum IgM and IgG were purified in a multi-step procedure using products from Thermo Scientific-Pierce (Rockford, Ill.). Sera samples (n=10 SCI, n=12 uninjured, 30-60 μL/mouse) were randomly selected and pooled to yield 510 μL total serum/group, 10 μL of which was reserved for determination of original Ig “purity” and concentration (see below). The remaining 500 μL samples were subjected to ammonium sulfate precipitation by adding 0.5 mL ice-cold saturated ammonium sulfate (SAS) dropwise over a 15-minute period. Proteins were allowed to precipitate overnight at 4° C., then were spun at 3000×g for 30 min and supernatants (including soluble protein contaminants) discarded. Precipitated proteins (including IgG and IgM) were resuspended in 0.5 mL Melon Gel Purification buffer, pH 7.0 (Thermo Scientific-Pierce), then residual ammonium sulfate was removed by dialyzing in four-400 mL volumes of the same buffer. Dialysis was performed in 20 kD molecular weight cut-off Slide-A-Lyzer Dialysis Cassettes (Thermo Scientific-Pierce) with continuous agitation via a stir bar. Dialysis buffer was completely replaced 3×, with the time in each volume being 1 hr, 2 hr, 14 hr, then 1 hr. After dialysis, an additional 10 μL was set aside for testing (see below) and the remaining volume was subjected to IgG purification using the Melon Gel Serum IgG purification kit (Thermo Scientific-Pierce) according to the manufacturer's instructions. After completion of the Melon Gel procedure, IgM was purified by eluting bound proteins from the Melon Gel support columns, then passing the remaining non-IgG “contaminants” through centrifugal filtration columns with a 100 kD molecular-weight cut-off. Purified IgG and IgM were combined and again filtered/concentrated in centrifugal filtration columns (100 kD molecular-weight cut-off). The retentate was resuspended in Melon gel purification buffer (pH 7.2) to identical concentrations (0.59 mg total IgM+IgG/mL) and stored at 4° C. until used for injection (within five days). Resulting samples were evaluated for Ig purity and concentration by SDS-Page/western blot and ELISA, respectively (see below and FIG. 9).

Verification of Antibody Purity

Verification of antibody purity was determined by SDS-Page and western blotting. For electrophoresis, 10 ng (1.69 μL) total purified protein and equal volumes of pooled, unpurified sera were used. Samples were separated by SDS-PAGE on 12% Bis-Tris gels (Invitrogen, Carlsbad, Calif.), then transferred to nitrocellulose membranes (parallel gels were run identically but total proteins were stained in-gel using Compasses' reagent) Immediately after transfer, membranes were stained for total proteins with Ponceau S, destained, then digitally photographed to visualize protein content. After blocking, membranes were probed with HRP-conjugated anti-mouse Ig (H+L, which detects IgM, IgG and IgA). Bound antibodies were detected by chemiluminescence (Millipore Immobilon Western HRP substrate) followed by exposure to autoradiography film (Kodak Biomax).

Determination of Antibody Concentration in Purified Samples

Concentration of total immunoglobulin (IgG and IgM) was determined using ELISA. Briefly, purified antibodies were diluted 1:100 in purification buffer and quadruplicate samples compared to a standard dilution series of purified mouse IgGI (Southern Biotech, Birmingham, Ala.).

Unilateral Antibody Microinjections

Equal volumes and concentrations of purified antibodies from SCI or uninjured mice were microinjected into the right ventral horn (T12 level) of naïve adult WT, C3^{-/-} or FcR^{-/-} mice. Under anesthesia and using sterile technique, a laminectomy was performed (with partial dural reflection) at the T₁₁₋₁₂ vertebral level. To ensure accuracy and to minimize pipette-mediated injury caused by respiratory movement, the spinal column was secured via the spinous processes adjacent to the laminectomy site using Adson forceps fixed in a spinal frame. Sterile glass micropipettes (pulled to an external diameter of 25-30 μm and pre-filled with either SCI or uninjured antibodies (0.59 μ.g/1 μl, dissolved in Pierce antibody purification buffer—a sterile, low-salt buffer solution, pH 7.2) was positioned about 0.3 mm lateral to the spinal midline using a hydraulic micropositioner (David Kopf Instruments, Tujunga, Calif.). From the meningeal surface, pipettes were lowered 0.9 mm into the ventral horn of the underlying gray matter. Using a PicoPump (World Precision Instruments, Sarasota, Fla.), 1 μl of purified antibody was injected over 15 minutes. Pipettes remained in place for two additional minutes to allow the injectate to dissipate into the parenchyma. To facilitate localization of the injection sites, the adjacent dura was marked with sterile charcoal. Postsurgical care, including closing of the surgery site, was performed as described for SCI.

Behavioral Evaluation of Antibody Microinjected Animals

Beginning one day after microinjection, mice were placed individually in an open field and then subjected to BMS testing (see above). However, because the injections were made into the right ventral horn and therefore affected the right hind limb only, the maximum score allowed for each animal was 5 (frequent or consistent plantar stepping, without forelimb-hindlimb coordination). After testing, 15-45 second video-clips were taken to further document behavioral differences.

### Experimental

### Tissue Processing and Anatomical Analyses

At 42 or 63 days after SCI or at 7 days after intraspinal microinjection, mice were anesthetized then transcardially perfused with 25 mL 0.1M PBS followed by 100 ml 4% paraformaldehyde in PBS. Brains and spinal cords were removed and post-fixed for 2 hours then stored in 0.2M phosphate buffer (PB) for 18 hours at 4° C. The next day, tissues were placed in 30% sucrose in 0.2M PB and stored for 48-72 hours. Sucrose-infiltrated tissues were rapidly frozen on powdered dry ice then stored at −80° C. Spinal cord segments spanning the lesion (1 cm, centered on the impact or injection site) were blocked and embedded in Tissue Tek OCT compound (Sakura Finetechnical Co., Tokyo, Japan), then molds were rapidly frozen with powdered dry ice. From these blocks, 10 μm coronal sections were cut on a cryostat and collected onto SuperfrostPlus slides (Fisher Scientific, Waltham, Mass.). SCI and microinjection lesion blocks were collected as 20 sets of serial sections (200 nm between adjacent sections on each slide) and stored at −20° C.

Histology and Immunohistochemistry

Adjacent sets of sections encompassing the lesions were stained with eriochrome cyanine (EC) plus cresyl violet (CV) or EC plus anti-mouse 200 kD neurofilament (chicken anti-NFH, see below). When primary antibodies were derived from mice, nonspecific staining was blocked using a cocktail of bovine serum albumin and complete horse and mouse serum for 1 hour at room temperature. After blocking solution was removed, sections were overlayed with pre-conjugated primary and secondary antibody cocktail (e.g., containing mouse anti-neurofilament plus biotinlyated horse anti-mouse IgG diluted in blocking solution). This antibody cocktail was incubated for 18 hours at room temperature or 4° C. In some cases, cell nuclei were revealed using DAPI or Draq5 (Biostatus Limited, UK). A list of primary antibodies and their final concentrations follows: rat anti-mouse Clq (Abcam, Inc., Cambridge, Mass.; clone 7H8, 0.133 ng/mL), rat anti-mouse CD45R/B220 (AbD Serotec, Oxford, UK; clone RA3-6B2, 0.83 μg/mL), mouse anti-mouse CD68 (AbD Serotec; clone FA-11, 1 μg/mL), chicken anti-mouse NFH (Ayes Labs,; 1 ng/mL), mouse-anti-MBP (Covance, Princeton, N.J.; clone SM194, mouse ascites, 1:40000), goat ant-mouse IgG (γ-chain specific, Southern Biotech; 1 μg/mL), goat anti-mouse IgG (H+L) F(Ab′)₂ fragments (Jackson ImmunoResearch Laboratories, Inc., West Grove, Pa.; 1 ng/mL), goat anti-mouse IgG F(Ab′)₂ fragments (γ-chain specific, Jackson ImmunoResearch Laboratories, Inc.; 1 μg/mL).

Quantitative Spared White Matter and Lesion Analyses

Images of EC+CV and EC+NFH-stained sections were digitized using a Zeiss Axioplan II Imaging microscope and an MCID 6.0 Elite system (InterFocus Imaging, Cambridge, UK). High-power digitized sections were printed and areas of spared white matter, spared gray matter, and lesion, were manually circumscribed. Spared white matter is defined as regions containing normal to near-normal densities of both EC and transversely-oriented neurofilament staining. Spared grey matter is defined as tissue containing normal gray matter cytoarchitecture with visibly healthy neuron profiles. Lesion is defined as regions lacking either spared white or gray matter. Uniform point-grids were placed randomly onto the print-outs and points falling within each area of interest were tallied and recorded. Reference areas for each section (e.g., tissue area) and total tissue volume were estimated in the same manner. Point tallies were converted into volume estimates using the formula: volume=T a/p·¹¹Eᵢ₋₁Pᵢ, where T equals the slice spacing, a/p equals the calculated area per point and ¹¹Eᵢ₋₁Pᵢ equals the sum of the points sampled. Areas per section for each region were calculated using the same formula with omission of the T multiplier.

Statistical Analyses

All data were collected and analyzed without knowledge of group identities. All statistical tests were performed using GraphPad Prism version 5.00 (GraphPad Software, San Diego, Calif.). Group means for most analyses were compared using student's t-test or ANOVA with Tukey's post-test. Two-way ANOVA with or without repeated measures and with Bonferroni's post-tests were used to compare data sets containing two factors (e.g., behavior and time, or area and distance). Significance was set at p<0.05.

Results: B Lymphocytes Impair Spontaneous Recovery of Locomotor Function After SCI

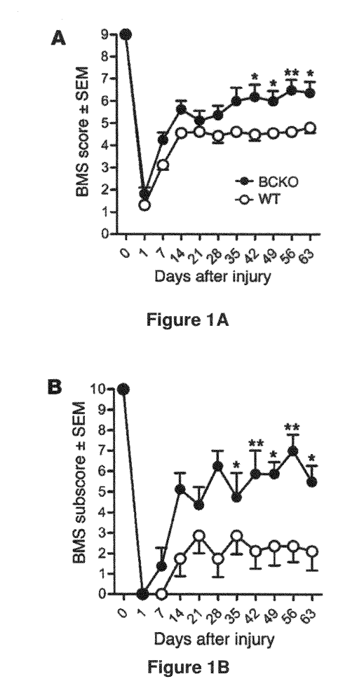

Mice with and without B cells received a SCI and locomotor recovery was evaluated for up to 9 weeks (FIG. 1A). Locomotor recovery plateaued in wild-type (WT) mice after two weeks with 35% (n=6/17) achieving fore-hind limb coordination by 63 dpi. Conversely, greater than 80% (n=13/16) of B cell knockout (BCKO) mice recovered bilateral weight-supported stepping within one week with additional recovery evident over the remaining 8 weeks. Ultimately, 88% (n=14/16; p<0.01 vs. WT mice) of BCKO mice recovered coordination with 41% (n=7/16) being nearly indistinguishable from uninjured mice; only subtle deficits in control of trunk or tail were visible. Refined aspects of hind limb usage also were improved with BCKO mice showing increased frequencies of fore limb-hind limb coordination, increased trunk stability and less medial or lateral rotation of the paws during the step cycle (FIG. 1B).

Locomotor function was analyzed using BMS (FIG. 1A) and subscore (FIG. 1B) analyses. The n=16-17/group is from two replicate studies giving equivalent results. *p<0.05, **p<0.01, vs. WT: Two-way ANOVA with repeated measures, Bonferroni post-test.

Results: Spinal Cord Pathology is Reduced in Mice Lacking B Cells

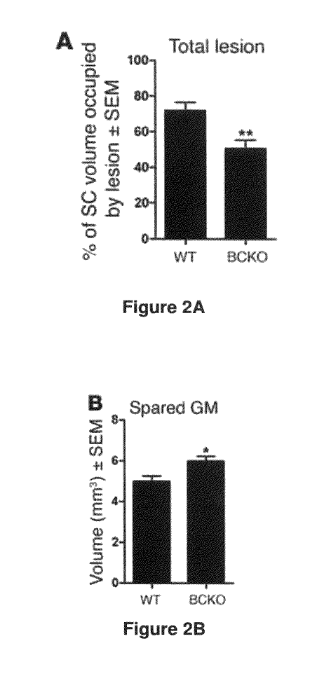

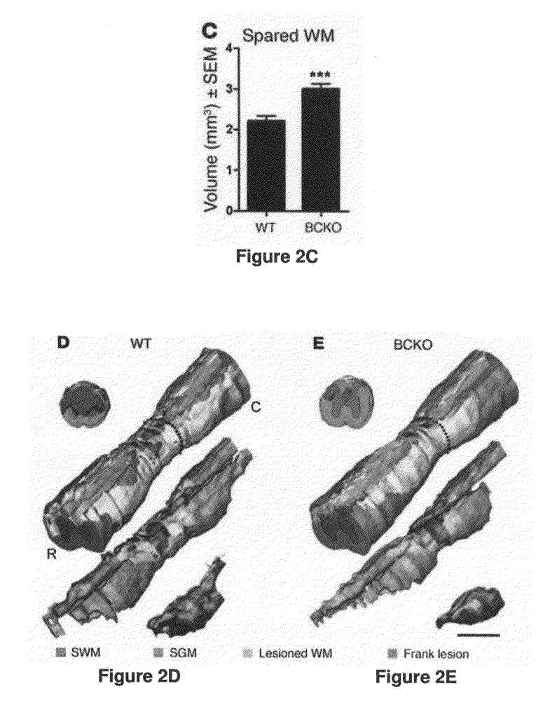

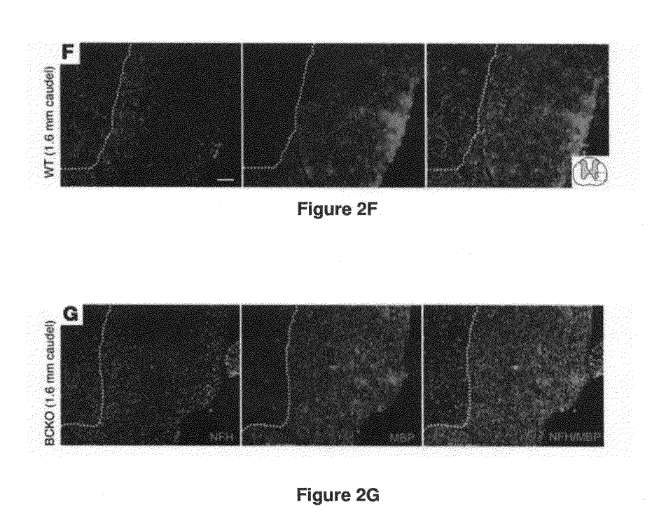

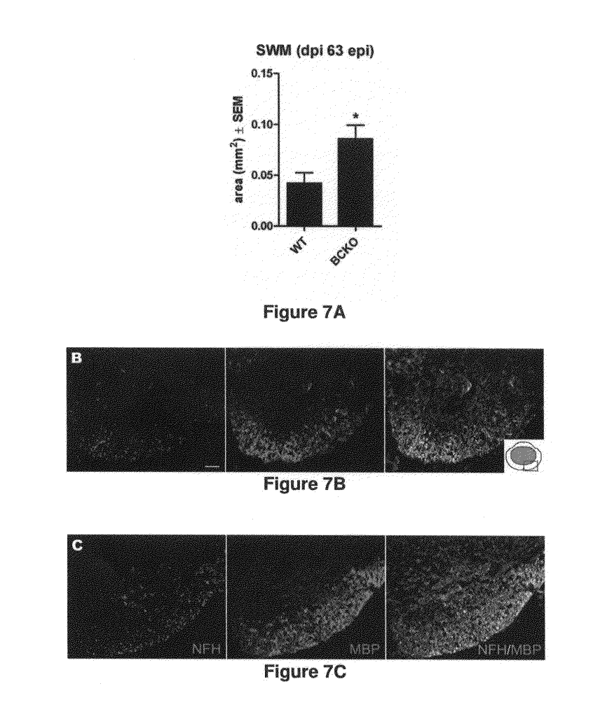

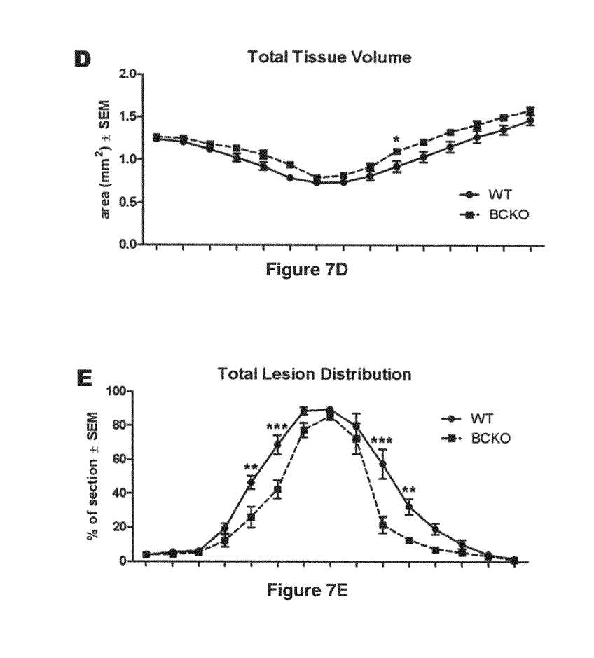

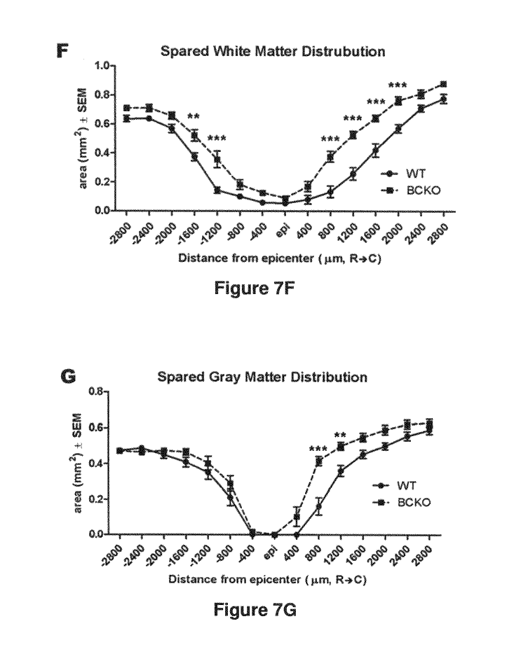

The lesion pathology caused by spinal cord contusion is characterized by a centralized core region with complete cell loss (frank lesion) and surrounding areas extending rostral and caudal to the impact site. Thus, unbiased stereology was used to quantify the volume of lesioned spinal cord at 9 weeks post-injury. In BCKO mice, lesion volume was decreased greater than 30% relative to SCI WT mice (FIG. 2A). This was accompanied by an increase in the total volume of spared gray matter and white matter in BCKO mice (FIGS. 2B-2E), exceeding that in WT animals by greater than 36% and 20%, respectively. These differences were greatest in sections proximal/distal to the impact site (see FIGS. 7A-7G). Exacerbated white matter pathology in WT mice was revealed by quantitatively larger regions devoid of myelin basic protein (MBP) and axons (FIGS. 2F-2G).

As shown in FIG. 2, significant neuroprotection is evident in the injured spinal cord of BCKO mice. The total lesion volume (FIG. 2A) is reduced in BCKO mice and is accompanied by significant sparing of spinal cord gray matter (GM) (FIG. 2B), and white matter (WM) (FIG. 2C). Volumes were estimated using Cavalieri's method. FIGS. 2D-2E illustrate three-dimensional reconstructions of spinal cords taken from animals with total lesion volume closest to the average for each group; gray equals spared white matter (SWM; regions containing myelin and axon profiles that are morphologically normal); green equals spared gray matter (SGM); red equals frank lesion (complete loss of normal cytoarchitecture); and yellow equals lesioned white matter (regions where axons and myelin are absent). Coronal slabs are sampled at 0.8 mm caudal to the injury epicenter and are marked by dashes in the complete 3D reconstructions (FIGS. 2D-2E). Immuno-fluorescent double-labeling of spared white matter 1.6 mm caudal to the injury epicenter from a WT (FIG. 2F) and BCKO mouse (FIG. 2G) reveals increased sparing of axons (green, anti-NFH) and myelin (red, anti-MBP) in BCKO mice. Dotted line delineates gray matter-white matter interface. Blue (DAPI) staining in merged image reveals cell nuclei. The schematic shown in top right panel (FIG. 2F) depicts imaged region. Scale bar in e=0.5 mm, f=40 nm; *=p<0.05, ***=p<0.001 vs. WT; 2-tailed t-tests. All data was collected at 63 dpi.

FIGS. 7A-7C show quantitative and qualitative analysis of spared white matter at the epicenter (epi) at 63 dpi Immunofluorescent double-labeling of representative images from WT (FIG. 7B) and BCKO (FIG. 7C) mice show axons (NFH staining) and myelin (MBP staining) in the epicenter (dashed box inset in FIG. 7B shows region where images were sampled); *=p<0.05, t-test. The rostral-caudal distribution of total tissue (FIG. 7D), total lesion (FIG. 7E), and spared white and gray matter (FIGS. 7F-7G, respectively) are further shown. Data in FIGS. 7D-7G were analyzed by two-way ANOVA with Bonferroni's post-test; *=p<0.05, **, p<0.01, ***=p<0.001.

Results: Antibody-Secreting B Cells (Plasma Cells) and Antibodies Accumulate in Cerebrospinal Fluid and Injured Spinal Cord

In SCI individuals, high levels of antibodies are found in CSF; however, the functional significance of these changes is unclear

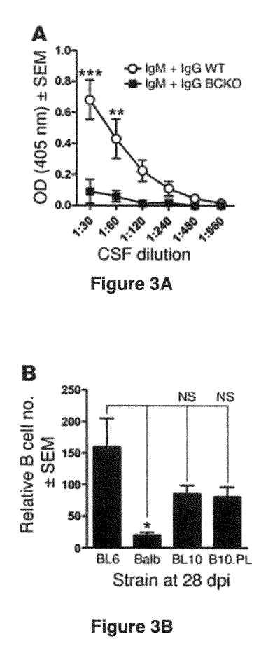

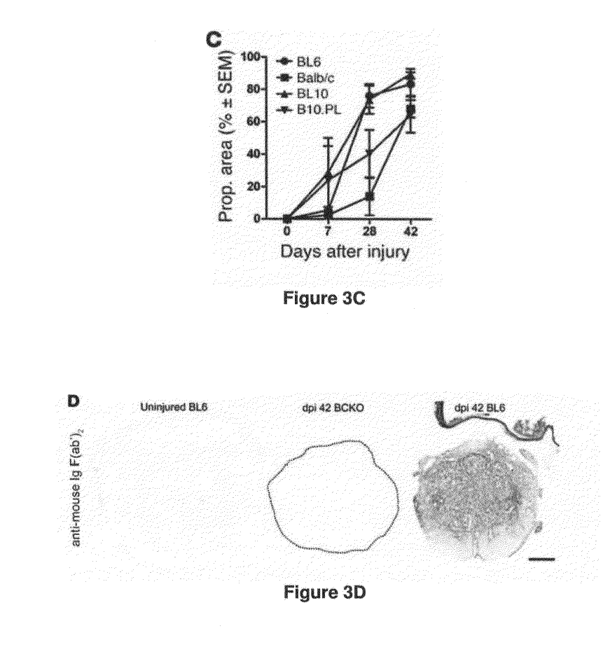

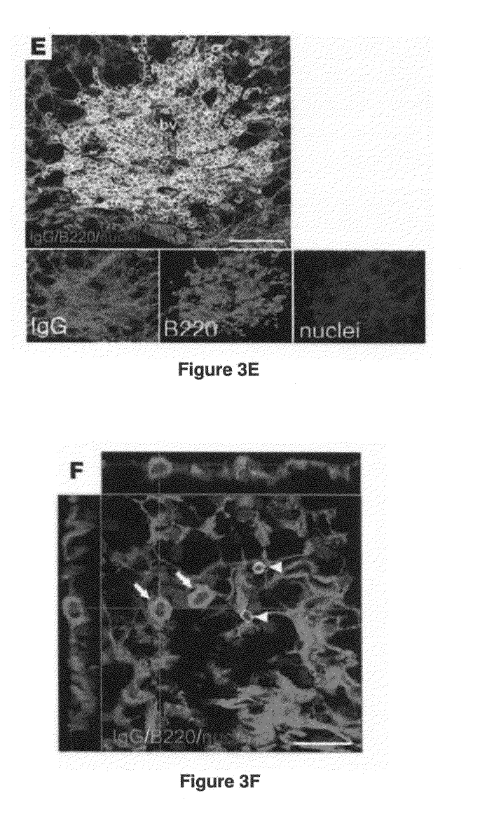

The inventors show for the first time herein that like human SCI, immunoglobulins (total IgM and IgG) are present in CSF of SCI WT mice but not in BCKO mice (FIG. 3A). To determine whether intraspinal B cell and antibody accumulation after SCI is unique to BL6 mice, injured spinal cord sections from different mouse strains with distinct immunological responses to SCI were analyzed, including C57BL/6 (WT mice), Balb/c, C57BL/10, B10.PL and BCKO mice (FIGS. 3B-3C). Staining with anti-B220 to mark mature B cells revealed the presence of intraspinal B cell infiltrates in all strains examined (FIG. 3B). To show that antibodies directly bind antigens in the injured spinal parenchyma, injured spinal cord sections were stained with F(Ab)₂ fragments of goat anti-mouse IgG (FIGS. 3C-3D). This allows visualization of antibodies and IgG-expressing B cells without non-specific labeling of cells expressing Fc receptors (e.g., microglia/macrophages). Sections from uninjured or SCI BCKO mice had negligible IgG-labeling (FIGS. 3C-3D). In contrast, intralesion antibody staining increased in all SCI mice as a function of time-post injury (FIGS. 3C-3D) indicating that SCI-induced activation of B cells and with enhanced antibody synthesis is not strain-specific.

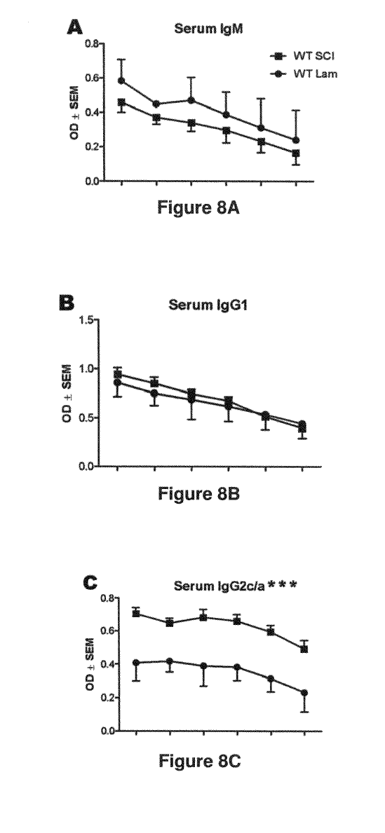

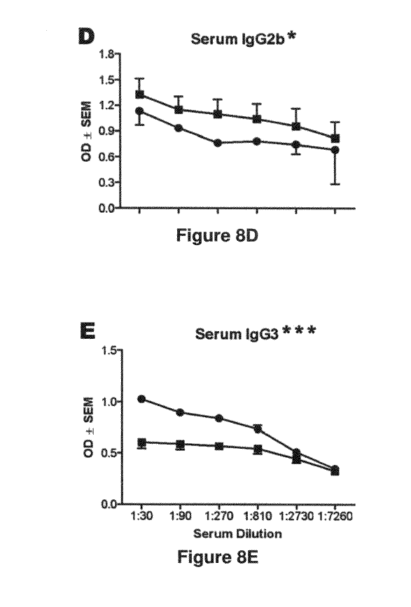

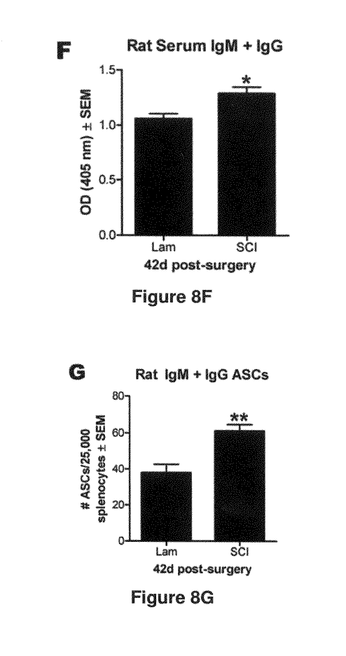

Levels of circulating (serum) IgM and IgG antibodies are increased in chronic SCI mice (see FIG. 8). Circulating antibodies were also increased after SCI in rats, indicating that SCI-induced B cell responses are not species-specific. Circulating immunoglobulins could cross the damaged blood brain barrier early after SCI or could accumulate in the CSF and spinal parenchyma via transcytosis. Moreover, the progressive increase in intraspinal antibody (FIG. 3C) in the face of decreasing blood-brain barrier permeability show that terminally differentiated, antibody-secreting plasma cells may also populate the injured spinal cord. The maturation state of intraspinal B cells was analyzed. In all mouse strains, B cells infiltrate the lesion site where they form dense cell clusters (FIGS. 3B and 3E). Most B cells are B220⁺ and IgG⁺, indicting they are activated but have not differentiated into antibody-secreting plasma cells (FIG. 3E). However, terminally differentiated B220⁻ plasma cells with intense, cytoplasmic IgG-labeling were prevalent in SCI lesions (shown for BL/6 mice; FIG. 3F).

As shown in FIG. 3, B cells and plasma cells accumulate in the CSF and injured spinal cord. In contrast to wild-type mice, BCKO mice fail to produce intrathecal antibodies (ELISA analysis of cerebrospinal fluid (CSF) from n=8 WT and BCKO mice) (FIG. 3A). FIGS. 3B and 3C illustrate a schematic graph of the quantitation of intraspinal B cell accumulation at 28 dpi (FIG. 3B) and the proportional area of IgG staining as a function of time post-SCI (FIG. 3C) at the site of injury in BL/6 (wild-type), Balb/c, C57BL/10 and B10.PL mice. The intraspinal accumulation of B cells and antibodies is not strain specific, only the magnitude varies; n=4-8 mice/strain; each bar in FIG. 3B represents the average of three sections spanning 600 μm centered on the injury site.

In FIG. 3D, representative sections from uninjured BL/6 or SCI BCKO (spinal cord circumscribed by dotted line) and WT mice (42 dpi) reveal the specificity of IgG labeling quantified in FIG. 3C, i.e., no labeling exists in spinal cord of uninjured or BCKO mice, scale bar in FIG. 3D equals 200 μm.

In FIG. 3E, flattened confocal z-stack image reveals accumulation of endogenous antibodies (green; Ig) and Ig+B cells in the injured spinal cord (42 dpi, individual color channels shown below in FIG. 3E).

FIG. 3F is a flattened z-stack image with x,y,z-projections showing B220-negative plasma cells with IgG⁻ cytoplasm (see arrows) co-localized with but distinct from IgG⁺B220⁺ B cells (see arrowheads). Scale bar in FIG. 3E equals 50 μm and in FIG. 3F equals 20 μm; ***=p<0.001, **=p<0.01 two-way ANOVA with Bonferroni post-test: *=p<0.05, one-way ANOVA with Tukey's post-test.

As shown in FIGS. 8A-8E, isotype-specific ELISA reveals differential production of different antibody isotypes at 63 dpi in C57BL/6 WT mice. Serum from BCKO mice produce OD values that are equivalent to blank wells (not shown). Sera dilutions are provided on x-axis in FIG. 8E; *=p<0.05, ***=p<0.001, two-way ANOVA comparing effect of surgery (Sham versus SCI, n=8/group). FIGS. 8F-8G illustrate ELISA data comparing total Ig levels in rat serum 42 days post-injury or sham injury (laminectomy (Lam) only); *=p<0.05, **=p<0.01 vs. Lam, (t-test; n=6 Lam, n=10 SCI).

Results: SCI-Induced Antibodies Cause Behavioral Deficits in Naïve Mice

Sera from SCI WT but not SCI BCKO or uninjured mice causes neuroinflammation and neuron death when microinjected into intact CNS. Antibodies, cytokines or other blood-derived factors produced after SCI may cause these changes. Since high levels of antibodies exist in the circulation and CSF after SCI, the inventors tested whether these byproducts of B cell activation could directly cause the pathology described previously.

Antibodies were purified from blood of control or SCI mice. FIGS. 9a and 9B illustrate the characterization of antibodies purified from sera of SCI mice. Identical volumes were loaded into each lane prior to SDS-PAGE and western blotting. FIG. 9A shows coomassie-staining of total proteins in the gel prior to transfer. FIG. 9B shows anti-mouse IgM+IgG staining of the membrane post-transfer.

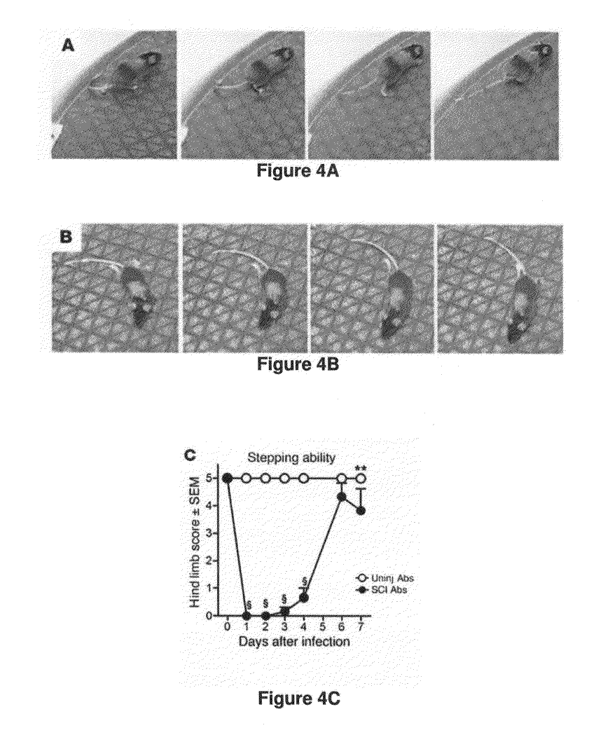

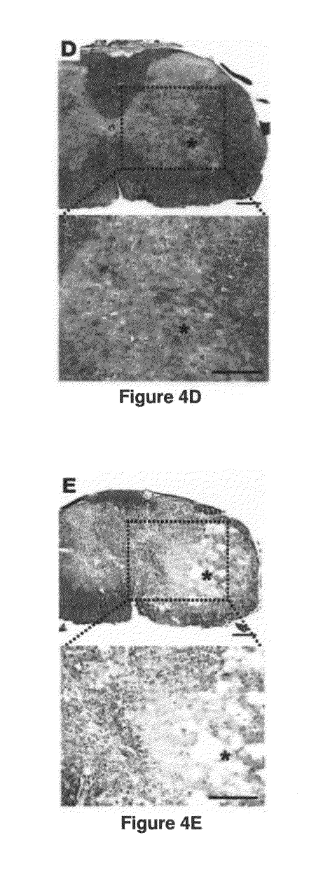

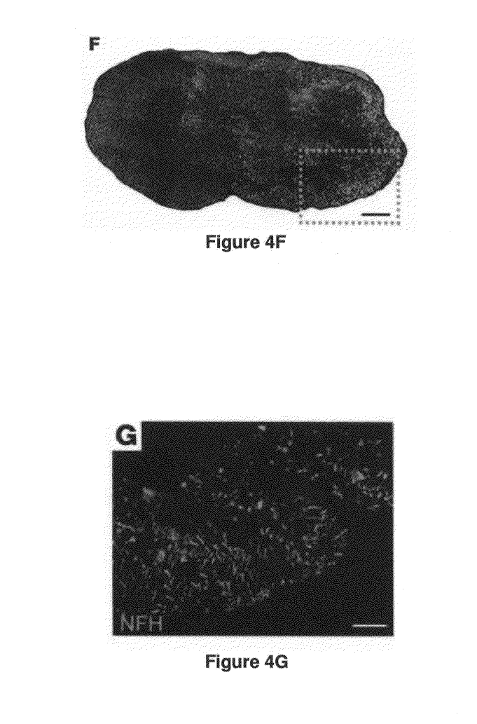

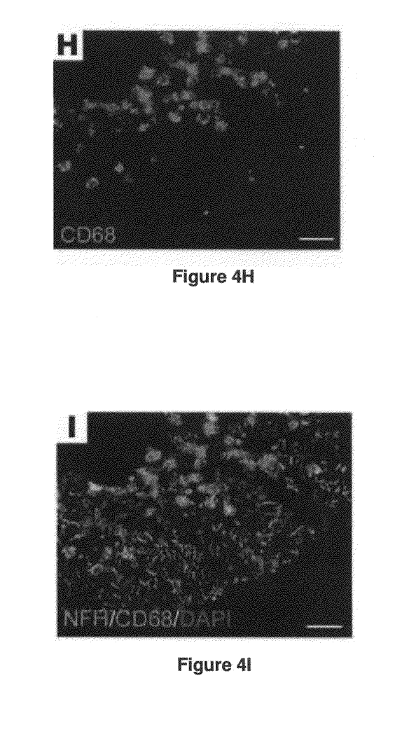

Purified antibodies were microinjected into the spinal cord of naïve mice at a concentration of ˜0.6 μg/μl, which is ˜ 1/10th the concentration of IgG in normal blood. Overground locomotor function was monitored in all mice for up to 7 days. Mice microinjected with antibodies from control mice (n=9) had no obvious gait deficits at any time post-injection (FIGS. 4A and 4C). In contrast, the hind limb ipsilateral to the injection site became paralyzed in all mice injected with SCI antibodies (n=6; FIGS. 4B-4C). Full paralysis was evident during the first 48 hours with varying degrees of recovery noted over the course of one week. Even by one week, mild to significant hind limb deficits persisted. Some mild functional impairment (e.g., dorsal stepping, toe drags) initially was visible initially in the contralateral hind limb but normal function was restored within 72 hours (not shown).

Unilateral intraspinal microinjection of antibodies purified from SCI mice causes hindlimb paralysis and neuropathology. FIGS. 4A and 4B show a sequence of still video images one injecting naïve (uninjured) mice with control (uninjured) (FIG. 4A) or SCI antibodies (FIG. 4B). One complete step cycle is depicted in both cases. FIG. 4C graphically depicts a summary of hind limb function ipsilateral to the site of injection. Scoring is based on 0-5 on BMS scale: 0=complete paralysis, 5=plantar stepping during greater than 50% of step cycles.

Results: SCI-Induced Antibodies are Neurotoxic—Anatomical Analyses

No visible pathology was present in spinal cords injected with control antibodies (FIG. 4D). In contrast, large necrotic inflammatory lesions occupied the ipsilateral gray matter and most of the white matter in all mice injected with SCI antibodies (FIGS. 4E-4F). Of note was the complete loss of neurons over a rostro-caudal distance of about 3.0 mm with some pathology evident across the midline in gray and white matter. Contralateral pathology was restricted to within about 2 mm of the injection site and likely contributed to the transient loss of function in the contralateral hind limb of some mice. In white matter on the injected side, axons were lost or extensively damaged and these regions co-localized with intense microglia/macrophage activation (FIGS. 4G-4I). Few T cells infiltrated sites of antibody-mediated pathology (not shown).

FIGS. 4D-4E show low and high power images from a mouse injected with control or SCI antibodies, respectively. Intraspinal pathology is only evident in mice receiving SCI antibodies; * indicates injection target. In FIG. 4F, phagocytic microglia/macrophages (red; anti-CD68) co-localize with axon/neuron pathology (green; anti-neurofilament 200 kD) at the site of injection in mice receiving SCI antibodies. Scale bars in FIGS. 4D-4F equal 0.2 mm FIGS. 4G-4I show high power images of boxed region in FIG. 4F. Scale bars in FIGS. 4G-4I equal 50 nm.

Results: SCI-Igs Cause Neuropathology via Complement- and FcR-Dependent Mechanisms

When antibodies bind antigen they form immune complexes (ICs) that cause tissue injury through activation of complement or recruitment/activation of cells bearing receptors for IgG, the Fc receptors. To determine if these mechanism(s) contribute to the pathology and loss of function caused by SCI antibodies, control or SCI antibodies were injected into the intact spinal cord of mice deficient in complement component C3 (C3^{-/-}) or the Fc receptor gamma chain (FcRγ^{-/-}).

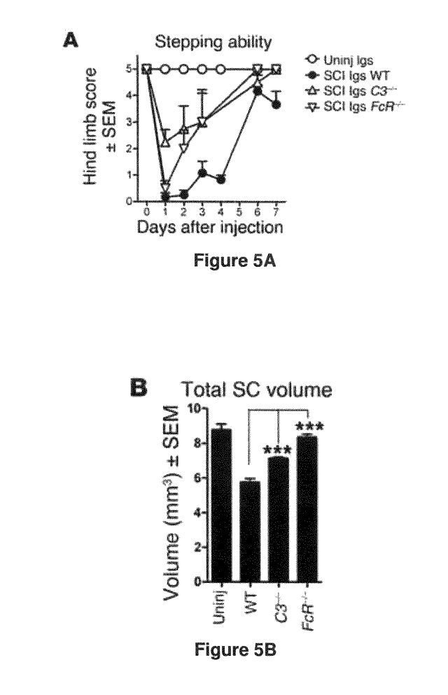

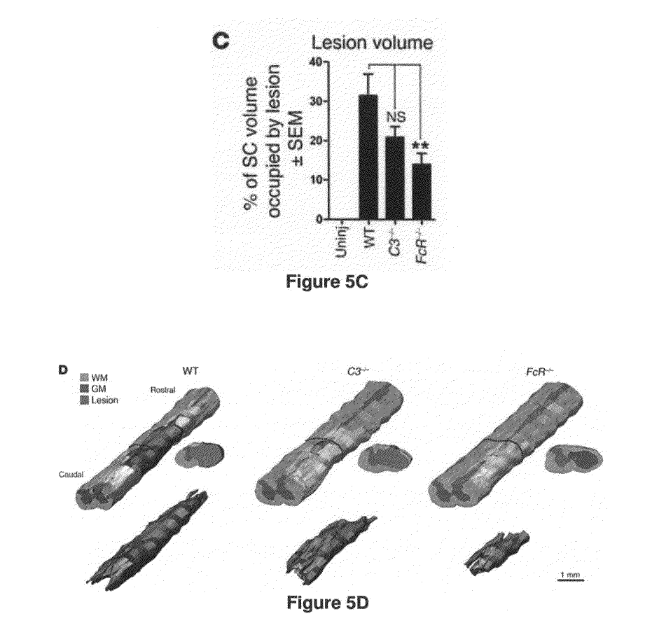

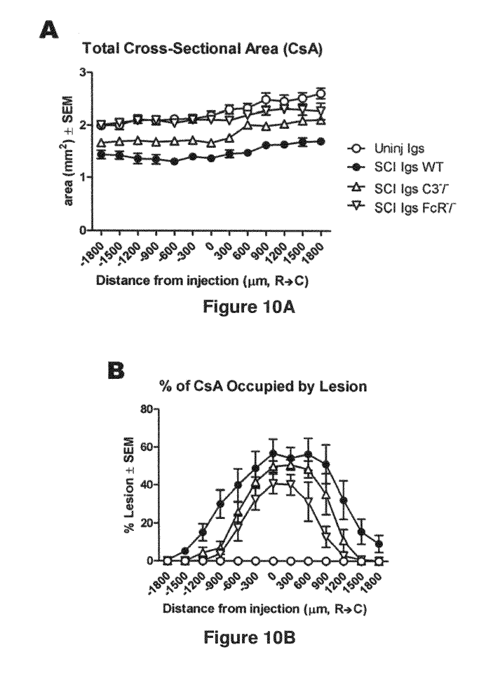

As before, SCI antibodies caused complete but transient paralysis when injected into WT mice (FIG. 5A). In contrast, hind limb deficits were attenuated in C3^{-/-} and FcRγ^{-/-} mice and the rate of spontaneous recovery was accelerated relative to WT mice (FIG. 5A). Stereological analyses of the spinal cord lesions from each mouse revealed significantly reduced pathology across the rostro-caudal axis of the spinal cord in C3^{-/-} and FcRγ^{-/-} mice (see FIGS. 5B-5D and FIGS. 10A-10B).

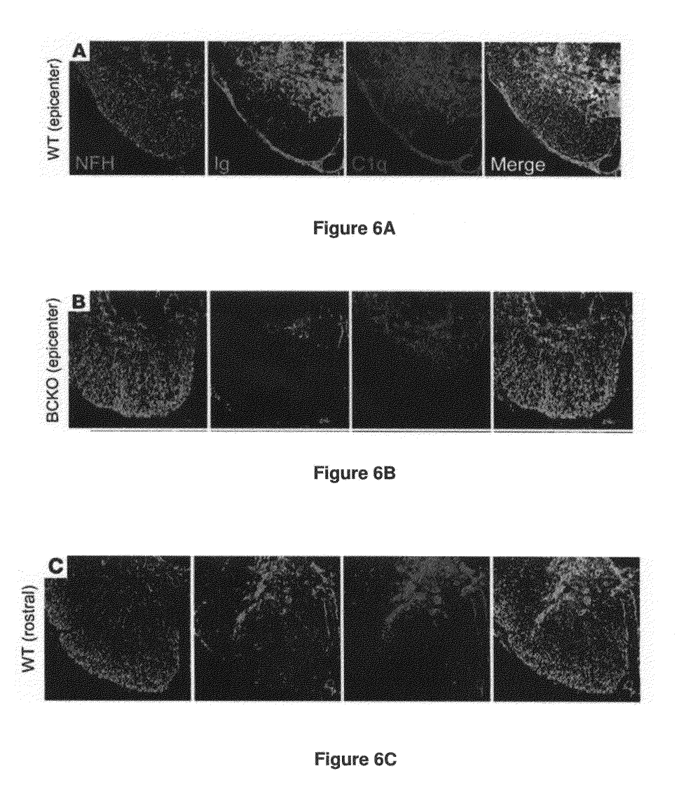

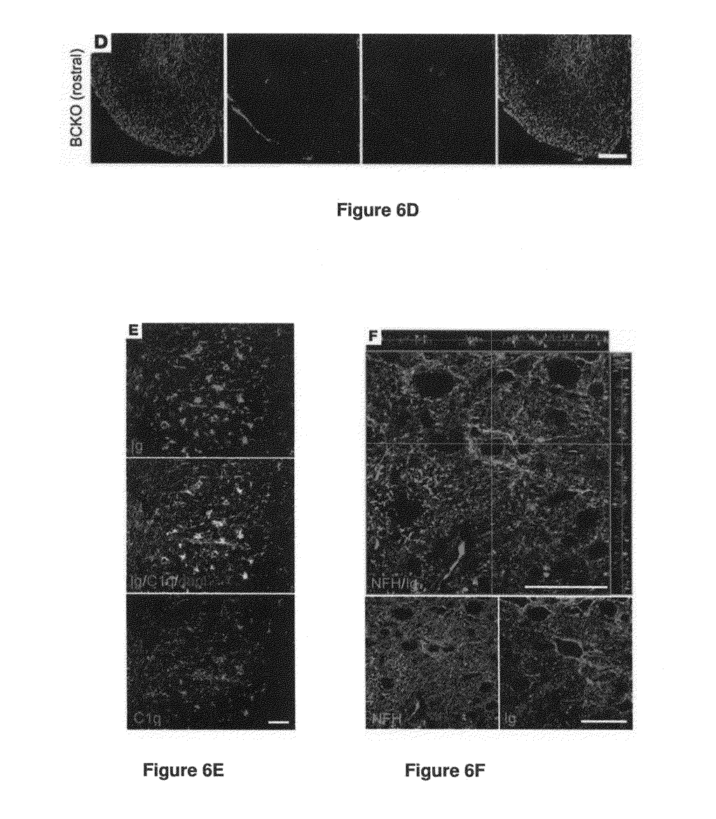

The latter data show that the ICs formed after SCI may cause injury to target cells in part through activation of complement. To examine if intraspinal antibodies co-localize with complement near or on putative target cells after SCI, confocal microscopic analyses of injured WT spinal cords were completed. In WT mice (FIGS. 6A, 6C, and 6E-6F), antibody/Clq deposits were prevalent and consistently decorated cells with endothelial, glial and neuron morphologies (FIGS. 6E-6F, endothelial labeling not shown). In contrast, minimal antibody/Clq labeling was found throughout injured BCKO spinal cords (FIGS. 6B and 6D).

SCI antibody-mediated neuropathology is complement and Fc-receptor dependent. FIG. 5A shows a summary of function in hind limb ipsilateral to the site where purified control or SCI antibodies were injected (see FIG. 4). Control or SCI antibodies were injected into wild-type (WT), complement deficient (C3^{-/-}) or FcγR^{-/-} mice. Referring to FIGS. 5B-5C, 5C1 antibodies cause marked pathology over ˜3.6 mm of spinal cord as in FIG. 4. This is significantly reduced in mice deficient in complement or Fe receptors. In FIG. 5D, three-dimensional reconstructions show the pathology caused by injections of SCI antibodies into WT, C3^{-/-} or FcR^{-/-} mice. Gray equals intact white matter; green equals intact gray matter; and red equals lesioned tissue. A spinal cord closest to the mean lesion volume is shown for each group (FIG. 5D).

IgG and complement Clq co-localize in regions of pathology in spinal cord of WT mice. As shown in FIGS. 6A and 6C, confocal microscopy reveals a relationship between axons (green, anti-NF200 kD), immunoglobulins (red, anti-mouse Ig) and complement Clq (blue, anti-Clq) in the ventrolateral funiculus at and rostral (1.6 mm) to a site of SCI in WT mice. As shown in FIGS. 6B and 6D, in BCKO mice, sparse Ig and Clq labeling can be seen among markedly preserved axon tracts and gray matter. FIG. 6E shows co-localization of IgG (green) and Clq on cells with glial morphology in the lateral funiculus ˜400 nm caudal to the epicenter. FIG. 6F shows x/y/z-projections of a flattened z-stack image from a section adjacent to site of injury showing IgG and NFH co-localization in the ventral horn on a cell with motor neuron morphology (center). Single channel images are depicted below in FIG. 6F. Scale bars equal 100 nm in FIGS. 6A-6D and 50 nm in FIGS. 6E-6F.

FIG. 10 shows the rostral-caudal distribution of total tissue (cross-sectional areas)

(FIG. 10A) and fraction of section occupied by lesion (FIG. 10B) in WT, C3^{-/-} or FcγR^{-/-} mice receiving microinjections of purified control (Uninj) or SCI antibodies; *=p<0.05, **=p<0.01, ***=p<0.001 via two-way ANOVA with Bonferroni's post-test.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof.

Therefore, it is intended that the invention not be limited to the particular embodiment disclosed herein contemplated for carrying out this invention, but that the invention will include all embodiments falling within the scope of the claims.

The publications and other materials used herein to illuminate the invention or provide additional details respecting the practice of the invention, are incorporated by reference herein, and for convenience are provided in the following bibliography.

Citation of the any of the documents recited herein is not intended as an admission that any of the foregoing is pertinent prior art. All statements as to the date or representation as to the contents of these documents is based on the information available to the applicant and does not constitute any admission as to the correctness of the dates or contents of these documents.

1. A method to ameliorate or reduce the risk of B cell-mediated spinal cord injury locomotor pathology in human or animal comprising administering a pharmaceutically effective dose of anti-CD20 antibody to a human or animal with spinal cord injury and ameliorating or reducing the risk of B-cell mediated spinal cord injury locomotor pathology.

2. The method of claim 1, wherein B cells are depleted via infusion of anti-CD20 antibodies.

3. The method of claim 2, wherein the anti-CD20 antibodies are selected from Rituximab or Ocrelizumab.

4. The method of claim 1, wherein B cells are depleted via infusion of a combination of anti-CD20 antibodies, anti-CD79alpha and anti-CD79beta antibodies.

5. (canceled)

6. (canceled)

7. (canceled)

8. (canceled)

9. (canceled)

10. (canceled)

11. The method of claim 1, wherein anti-CD20 antibody is administered in a dose of 1 mg to 1 g, preferably 100 mg to 800 mg, more preferably 250 mg to 750 mg, most preferably 300 mg to 500 mg.

12. The method of claim 1, wherein the anti-CD20 antibody is administered in one dose every 2-20 days, preferably one dose every 7-14 days.

13. The method of claim 1, wherein the anti-CD20 antibody is administered in one dose every 1-3 days.

14. The method of claim 1, wherein the anti-CD20 antibody is administered in 1-20 doses in total, preferably in 1-10 doses, more preferably 1-8 doses, and most preferably 1-4 doses in total.

15. The method of claim 14, wherein the administration is systemical, preferably via injection or infusion, more preferably an intravenous injection or infusion.

16. The method of claim 14, wherein the anti-CD20 antibody is administered to a subject in need thereof, and the administration results in a prevention of a deterioration of neurological function.

17. The method of claim 14, wherein the anti-CD20 antibody is administered prior to or after a different treatment modality.

18. The method of claim 14, wherein the anti-CD20 antibody is administered in combination with other medication.

19. A method for treating a patient with a neurological disorder, comprising:
providing a therapeutic B lymphocyte (B cell) depleting antibody to block B cell-mediated pathology in the patient; and
depleting B cells via infusion of antibodies in the patient to lessen the severity of tissue damage and to restore locomotor function in the patient.

20. The method of claim 19, wherein B cells are depleted via infusion of anti-CD20 antibodies.

21. The method of claim 20, wherein the anti-CD20 antibodies are selected from Rituximab or Ocrelizumab.

22. The method of claim 19, wherein B cells are depleted via infusion of a combination of anti-CD79alpha and anti-CD79beta antibodies.

23. A method of treating a neurological disorder, comprising administering to a subject in need thereof effective amounts of an anti-CD20 antibody, wherein administration of the anti-CD20 antibody provides a synergistic improvement in the incidence or symptoms of a neurological disorder.

24. The method of claim 23, wherein the anti-CD20 antibody is a non T-cell depleting antibody.

25. The method of claim 23, wherein the anti-CD20 antibody is a humanized antibody.

26. A method of treating a subject suffering from or predisposed to a neurological disorder comprising the step of:
administering a therapeutically effective amount of at least one B cell depleting antibody to the subject.

27. The method of claim 26, wherein the B cell depleting antibodies are monoclonal antibodies.

28. The method of claim 27, wherein the monoclonal antibodies are selected from the group consisting of chimeric antibodies and humanized antibodies.

29. The method of claim 26, wherein the neurological disorder is selected from the group consisting of traumatic brain or spinal cord injuries, spinal ischemia, stroke, Alzheimer's disease, Parkinson's disease, and schizophrenia.

30. The method of claim 26, wherein the B cell depleting antibody reacts with or binds to a CD20 antigen.

31. The method of claim 26, wherein the B cell depleting antibody is Rituximab and/or Ocrelizumab.

32. A method of treating a subject suffering from or predisposed to a neurological disorder comprising: administering a therapeutically effective amount of at least one immunoregulatory antibody to the subject, wherein the immunoregulatory antibody binds to an antigen selected from the group consisting of CD79alpha, CD79beta and CD20 antigens.

33. The method of claim 32 wherein the immunoregulatory antibody comprises a monoclonal antibody.

34. The method of claim 33, wherein the monoclonal antibody is selected from the group consisting of chimeric antibodies and humanized antibodies.

35. The method of claim 32, wherein the neurological disorder is selected from the group consisting of traumatic brain or spinal cord injuries, spinal ischemia, stroke, Alzheimer's disease, Parkinson's disease, and schizophrenia.

36. The method of claim 32, further comprising the step of administering a B cell depleting antibody.

37. The method of claim 32, wherein the B cell depleting antibody reacts with or binds to CD20 antigen.

38. The method of claim 32, wherein the B cell depleting antibody reacts with or binds to CD79alpha antigen.

39. The method of claim 32, wherein the B cell depleting antibody reacts with or binds to CD79beta antigen.

40. The method of claim 32, wherein the B cell depleting antibody reacts with or binds to a combination of CD79alpha and CD79beta antigens.
